(19) United States
(12) Patent Application Publication
(10) Pub. No.: US 2005/0000523 A1
(43) Pub. Date: Jan. 6, 2005

(54) Introducer and perforator guide for placing a tape in the human body

(75) Inventor: Jean-Marc Beraud, St. Etienne (FR)

Correspondence Address: DENNISON, SCHULTZ, DOUGHERTY & MACDONALD, 1727 KING STREET, SUITE 105, ALEXANDRIA, VA, 22314, US

(73) Assignee: Analytic Biosurgical Solutions- ABISS

(21) Appl. No.: 10/809,799

(22) Filed: Mar. 26, 2004

(30) Foreign Application Priority Data
Mar. 28, 2003 (FR) FR 03 03 897

(51) Int. Cl. A61B 17/10

(52) U.S. Cl. 128/885; 606/139

(57) ABSTRACT

A device for placing a reinforcing tape in a tissue of the human body includes an introducer having an elongated flexible body, at each of both ends of the body, pulling means, between both ends, a cavity for receiving the tape, and, at the cavity, means for cutting the body into two portions separable by pulling exerted on the pulling means. The cutting means includes at least one aperture provided in the wall of the cavity and intended for the passage of a cutting tool. This device is applicable to the treatment of cystoceles.

The present invention relates to the technical field of inserting reinforcing tapes for strengthening impaired tissues of the human body.

In a preferred application, the invention relates to the field of reinforcing tapes used in uro-gynaecological surgery for treating stress urinary incontinence in women.

It is known how to treat stress urinary incontinence in women by means of a supporting tape which is placed under the urethra, so as to provide support to it.

For this purpose, patent application FR 2 804 010, for example, proposed the implementation, as a supporting component, of a tape in a woven or knitted biocompatible material, such as for example knitted monofil polypropylene. This tape is then placed under the urethra of the patient to be treated, by means of an introducer which has an elongated flexible body provided at each of its both ends with two pulling means which define between their ends, an impervious receiving cavity inside which a reinforcing tape is positioned. According to this document, the cavity and the body of the introducer are made by assembling two half-bodies by means of a linking component having an area of weaker strength, as cutting means, capable of breaking up under the effect of joint pulling on the means for pulling ends of the body.

With such an introducer, it is actually possible to ensure the placing of the reinforcing tape, but however it has the drawback of being particularly difficult to cut, insofar as it is necessary to exert a large pull in order to tear the body of the introducer at its cutting means, and in the eventuality that it was not possible to provide this spontaneous cut by simple pulling, it is necessary to resort to a cutting tool with the risk of damaging the reinforcing tape.

Moreover, having to exert a large pull on the introducer has the risk of pre-stressing the reinforcing tape, so that the latter excessively raises the urethra of the patient, thereby preventing either a complete emptying of the bladder, or any emptying of the bladder, thus imposing a further corrective surgical operation.

Thus, the need appears for a new device for placing a reinforcing tape which provides cutting means which are easy to implement and which guarantee a laying of the tape in situ without any pre-stresses.

To achieve this goal, the invention relates to a device for placing a reinforcing tape in a tissue of the human body, comprising an introducer which has:
an elongated flexible body,
at each of the two ends of the body, pulling means,
between both ends, a cavity for receiving the tape,
and, at the cavity, means for cutting the body into two portions separable by pulling exerted on the pulling means.

According to the invention, the placing device is characterized in that the cutting means comprise at least one aperture provided in the wall of the cavity and for the passage of a cutting tool.

The implementation of such an aperture enables the body of the introducer to be cut, at the cavity for receiving the tape, therefore without any risks of cutting into the latter or damaging it, to the extent that this cutting is performed through the aperture, providing the surgeon with visual control of the position of the tape inside the cavity.

According to the invention, the cutting means comprise at least one or more apertures and according to one embodiment, the cutting means comprise at least two apertures positioned facing each other.

According to another feature of the invention, the aperture(s) is(are) positioned so as to allow placing of the tape in the cavity of the introducer.

This particularly advantageous feature enables the surgeon to select the type of reinforcing tapes which he will use at the very moment of the operation.

Thus, the surgeon may then choose to either implement a tape in a biocompatible synthetic material or, on the contrary, a tape in a biocompatible natural material, such as for example, “fascia latta” or even, a product sold under the brand PELVICOL.

According to another feature of the invention, the wall of the cavity has a series of perforations which allow recourse to sterilization of the introducer and of its possible content with steam.

According to another feature of the invention, the placing device comprises a tape positioned inside the cavity of the introducer, which is free, i.e., has no positive mechanical linkage with the introducer, and more particularly with its receiving cavity, so that stresses applied to the introducer are not transmitted to this tape.

According to the invention, the pulling means of the introducer may be made in a suitable way and may be removable or not.

According to a preferred embodiment, the pulling means comprise semi-rigid or semi-flexible needles integral with the ends of the elongated body of the introducer.

The introducer according to the invention may be used for placing a reinforcing tape according to the different known methods and procedures such as described for example but not exclusively, in Patent Application FR 2 804 010, or US patent application 2002099260.

In order to facilitate the work of the surgeon, according to another feature of the invention, the placing device also comprises at least one ancillary which comprises an elongated perforator guide or trocar, one end of which is intended to be introduced into the body of the patient and the other end of which is provided with a handle.

According to a feature of the invention, the perforator guide has an arcuate shape in a plane. Preferably but not strictly necessary, the arcuate portion of the perforator extends over an angular sector larger than 140° and preferably but not necessarily, less than 180° and preferably between 150° and 170°. Preferably, the arcuate portion of the perforator guide then has a radius of curvature between 30 mm and 60 mm, and preferably between 40 mm and 50 mm, for the portion of the perforator guide extending between the handle and the end, intended to be introduced into the body of the patient, the extreme portion of the perforator then having a variable radius of curvature.

According to another embodiment, the perforator guide has a helicoidal shape at its end opposite to the handle, or distal end. Preferably, the perforator guide then has a shape of a portion of a helicoidal coil extending over an angle between 180° and 360°, and preferably between 255° and 270°. Also, preferentially, the coil of the perforator guide has a radius of curvature between 20 mm and 40 mm, with a pitch between 15 mm and 25 mm.

According to another feature of the invention, in order to reduce traumas suffered by the body of the patient upon introduction of the implant, the introducing device further comprises a tubular sleeve with a shape complementary to that of the perforator guide. This tubular sleeve is then intended to be engaged onto the perforator guide and to remain in the body of the patient after removing the perforator guide in order to define a tunnel for the passage of the pulling means of the introducer. The tubular sleeve is then removed, after passing the pulling means through, upon removing the introducer.

According to the invention, the tubular sleeve may be made in any biocompatible flexible material, such as for example, but not exclusively, PVC.

According to the invention, the tubular sleeve may either have a length substantially equal to that of the useful portion of the perforator guide, or a larger length than the useful length of the perforator guide. In this last case, the tubular sleeve will have a side aperture provided at a distance from a free end of the lower sleeve or equal to the length of the useful portion of the perforator guide, so as to allow the perforator guide to be placed in the sleeve while letting the tip of said perforator jut out of the sleeve.

Various other features of the invention will be apparent from the description below, made with reference to the appended drawings which illustrate different embodiments of an introducer according to the invention, as well as perforator guides for facilitating the implementation of the introducer according to the invention.

Moreover, it should be noted that the different features of the invention, as described previously and hereafter, may be combined together according to different alternatives, depending on the pathology to be treated.

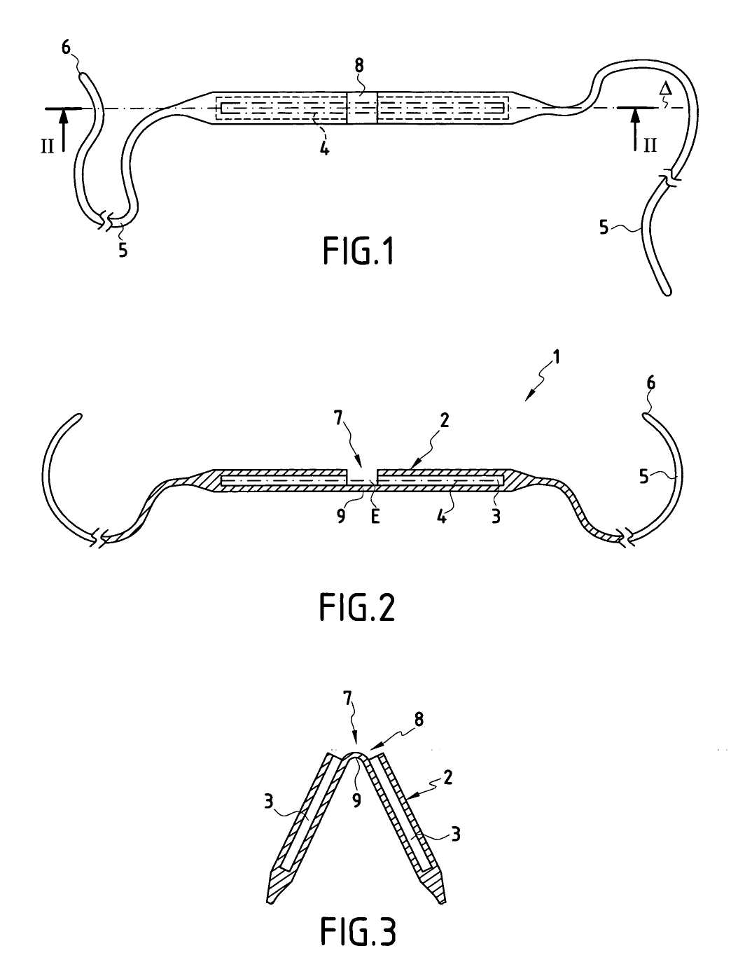

FIG. 1 is a top view of an introducer for placing a reinforcing tape according to the invention.

FIG. 2 is a partial section along line II-II of FIG. 1.

FIG. 3 is a section, analogous to FIG. 2, showing the introducer according to the invention, in a folded position, so as to allow a tape to be introduced into the cavity for receiving the introducer.

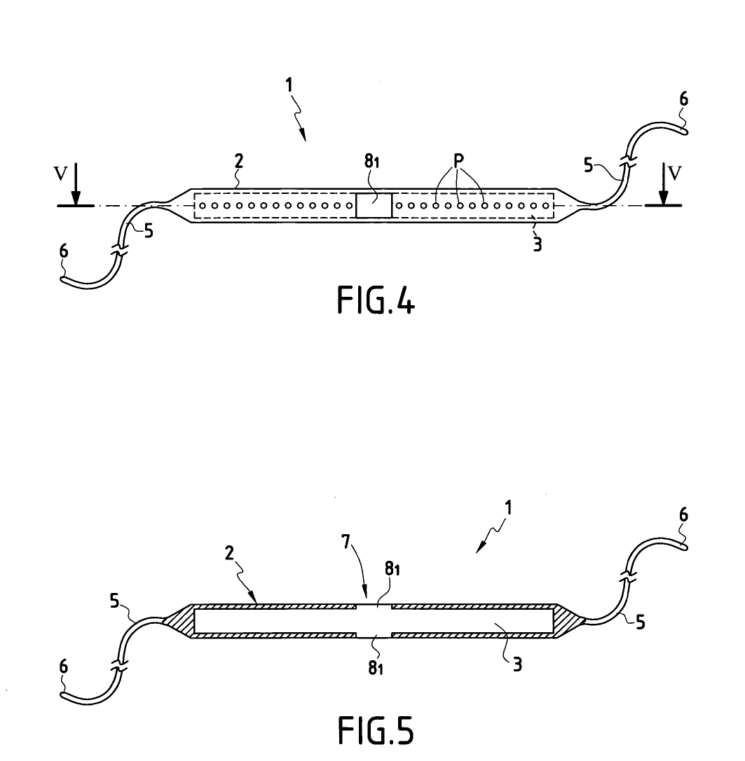

FIG. 4 is a side view of another embodiment of an introducer according to the invention.

FIG. 5 is a partial section along line V-V of FIG. 4.

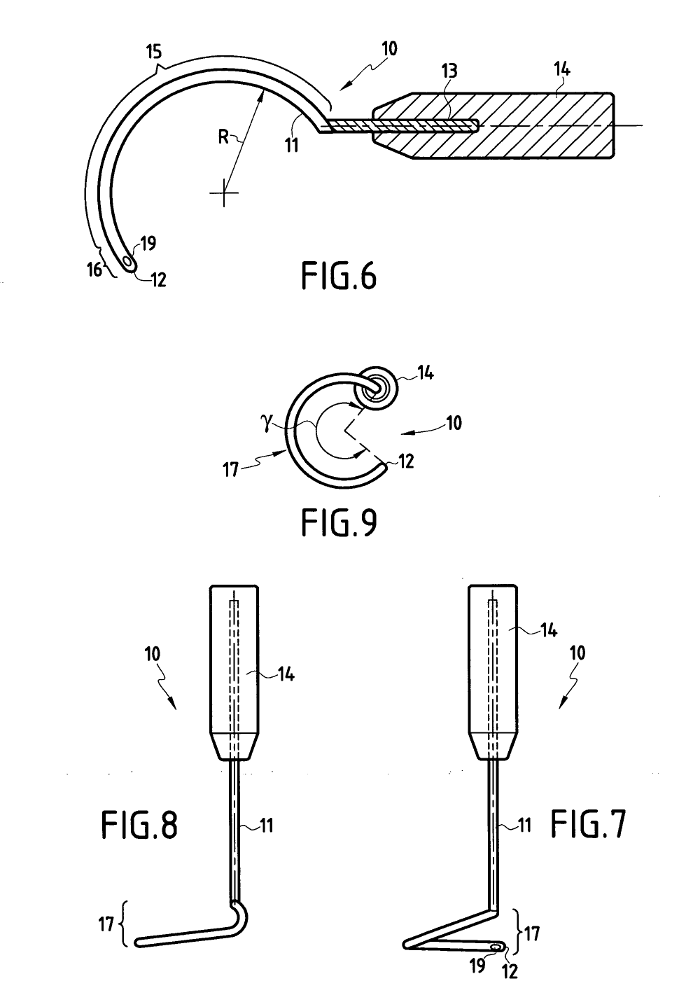

FIG. 6 is an elevation, partly pulled out, of a perforator guide which may be used for placing the implant according to the invention and having an arcuate shape.

FIG. 7 is an elevation of another embodiment of a perforator guide according to the invention, having an introduction end with a helicoidal shape.

FIG. 8 is a left view of the perforator guide according to FIG. 3.

FIG. 9 is a bottom view of the perforator illustrated in FIG. 3.

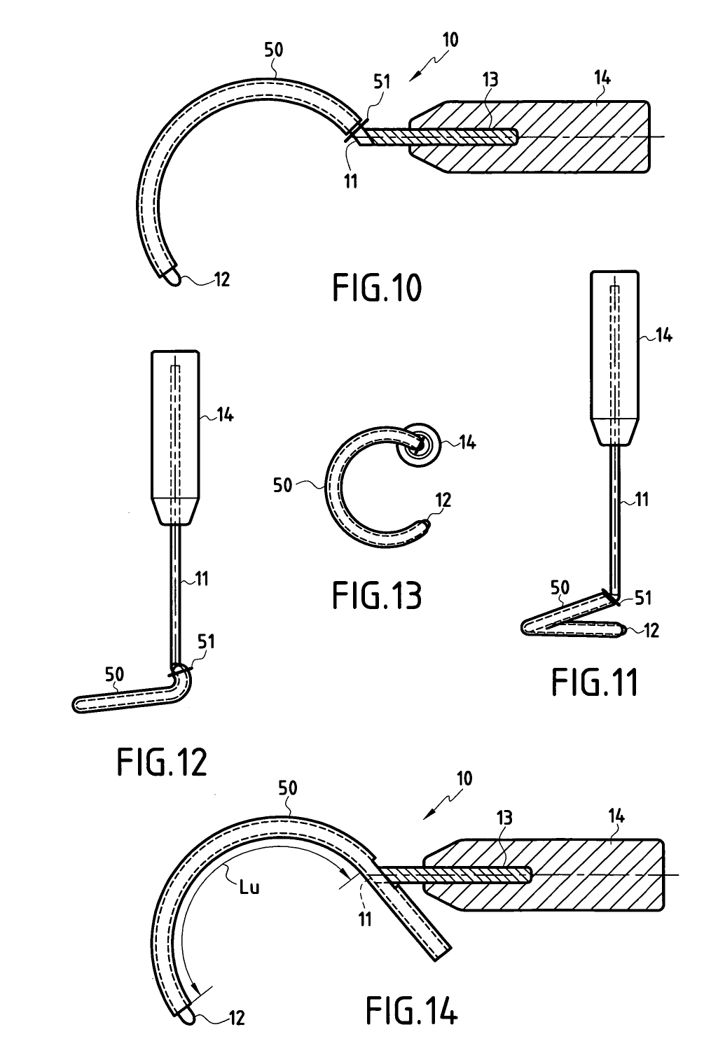

FIGS. 10-13 are views, analogous to FIGS. 6-7, showing alternative embodiments of perforator guides for placing an implant according to the invention.

FIG. 14 is an analogous to FIG. 10 further showing another alternative embodiment of a perforator guide according to the invention.

The invention is directed to providing means for facilitating the work of a surgeon for placing a supporting tape, used for the treatment, for example but not exclusively, of stress urinary incontinence.

For this purpose, the invention proposes a device for placing such a tape which first of all comprises an introducer, as illustrated in FIGS. 1 and 2 and designated in its whole by reference 1.

Such an introducer 1 comprises an elongated flexible body 2 which defines a cavity 3 for receiving a reinforcing tape 4, schematically materialized in dot and dash lines. The introducer 1 comprises at each of both ends of the body 2, pulling means 5 which may be made in any appropriate way, either removable or not.

According to the illustrated example, the pulling means 5 are formed, for each end of the flexible body, by a semi-rigid or semi-flexible needle having an introduction end 6 produced in the shape of a blunt tip, i.e., atraumatic, not liable to cut or injure the tissues in which it should be introduced.

The flexible body 2 and the pulling means 5 may be made in any compatible material and preferably in a synthetic polymer material from the family of low friction plastics, such as for example polyethylene. Preferably, the needle 5 will then be made in the same material as body 2, but that this feature may be considered as strictly necessary for making the introducer 1 according to the invention.

According to an essential feature of the invention, the introducer 1 finally comprises cutting means 7 which comprise at least one, and according to the illustrated example, exactly one aperture 8, provided in the flexible body 2 at the cavity 3. According to the illustrated example, the aperture 8 constituent of the cutting means 7, extends transversely to the longitudinal axis Δ of cavity 3 and affects more than half of the circumference of the wall of the cavity so as to only leave a connecting wall 9 between both portions of body 2 delimited by the aperture 8.

With this feature of the invention, the surgeon may cut the wall of the half-body 2 by coming and placing the tip of a cutting tool, such as a pair of scissors, in the gap E between the tape 4 and the wall 9, and is able to check the exact position of the tape 4 and therefore not to risk accidentally cutting the latter.

Moreover, this particular embodiment of the cutting means allows the tape to be folded at the wall 9, as illustrated in FIG. 3, so as to be able to place any type of tape in the cavity 3, according to the pathology to be treated. Thus, the introducer 1 according to the invention is not necessarily supplied to the surgeon with the reinforcing tape 4 positioned inside cavity 3.

The introducer device according to the invention may thus be used for any type of tapes in synthetic material or in natural material.

However, according to a feature of the invention, the introducer 1 comprises a tape pre-positioned in cavity 3 and being free with respect to the walls of the latter, so that the tensile stresses applied on the introducer 1 are not transmitted to the tape which it contains, so that the latter may be laid in a relaxed condition without any prestress.

According to the example illustrated in FIGS. 1-3, the cutting means 7 are formed by a unique aperture 8 provided in the wall of the body 2 at the cavity 3. However, such an embodiment of the cutting means 7 is not strictly necessary for making an introducer 1 according to the invention.

Thus, FIGS. 4 and 5 illustrate another embodiment of an introducer 1 according to the invention, for which the cutting means 7 are formed by two apertures 8, 81, provided in the body 2 at the cavity 3, so as to be facing each other. Moreover, according to this exemplary embodiment, the introducer 1 has micro-perforations P provided in the wall of cavity 3 so as to allow the inside of the latter and its possible content to be sterilized.

The implementation of an introducer 1 according to the present invention further has the advantage that the abrasion of the muscular tissues crossed during the placing of the tape 4 may be maximally reduced.

In the same sense, in order to reduce the dissection of the region of placement of the tape to a minimum and so the ensuing trauma, the invention proposes to the surgeon proceeding with the treatment, the use of one or more elongated perforator guides 10, such as those more particularly illustrated in FIGS. 6-9.

Generally, such a perforator guide 10 comprises an elongated body or mandrel, and the end 12 of which is intended to be introduced into the body of the subject to be treated, and the other end 13 of which is provided with a handle 14. It should be noted that the introduction end 12 is preferably formed by a blunt tip, i.e., an atraumatic tip which is not liable to injure or cut the tissues into which it has to be introduced.

According to an embodiment illustrated in FIG. 6, the perforator guide 10 has an arcuate shape in a plane. This arcuate shape in a plane is more particularly adapted to the placing of suspension slings in the upper and lower areas of the closed holes. Preferentially, but not strictly necessarily, the arcuate portion of the perforator guide then has a radius of curvature R between 30 mm and 60 mm, and preferably between 14 mm and 50 mm for the portion 15 of the perforator guide 10 extending between the handle 14 and the end 12, the extreme portion 16 of the perforator guide 10 then having a variable radius of curvature.

According to another embodiment of the perforator guide 10, illustrated in FIGS. 7-9, the elongated body 11 of guide 10 has an end 17 with a helicoidal shape, also suitable for placing suspension slings in the upper or lower areas of the closed holes. Preferentially, the distal end 17 of the perforator guide then has the shape of a portion of a helicoidal coil extending over an angle γ between 180° and 360°, and preferably between 255° and 270°. Also, preferentially, the coil 17 of the perforator guide has a radius of curvature between 20 mm and 40 mm, with a pitch between 15 mm and 25 mm.

It should be noted that according to these exemplary embodiments, the perforator guides 10 have at their ends 12 an eye 19 allowing fixation of needles 5 for providing pulling of the introducer 1 into the tissues where the tape 4 is to be placed.

However, the presence of such an eye 19 is not strictly necessary for making a perforator guide according to the invention.

Thus, in an attempt to maximally reduce the trauma by abrasion of the crossed tissue areas, the implementation of an ancillary or placing device may be contemplated, associating the perforator guide 10 with a flexible sleeve 50 with a complementary shape to that of guide 10 as this is illustrated in FIGS. 10 and 11-13. The sleeve 50 is engaged onto the perforator guide 10 which then has an abutment or guard 51 against which the sleeve 50 presses, upon introducing the perforator guide 10 into the body of the patient. Sleeve 50 is left in place in the body of the patient after removing the perforator guide 10 before the placement of the tape 4 and the passage of the introducer 10. The sleeve used thereby allows a channel to be generated for the passage of a pulling element 5 of the introducer 10 and wherein the needle 5 may be slidably displaced, so as to adjust the position of the tape 4 without abrading the tissues crossed. A sleeve 50 is then used for placing both ends of the tape 4. The sleeves 50 are then removed at the same time as the corresponding parts of the introducer 10.

Thus, implementing the sleeves 50 avoids acute inflammatory phenomena and reduces the tissues' trauma, insofar as the implantation sites consist of very specialized muscular tissues which have lost a large portion of their regeneration and fast healing capacities.

According to the examples illustrated in FIGS. 10-13, the flexible sleeve 50 has a length substantially equal to the useful length of the perforator guide, i.e., the portion of the latter between its tip and the abutment or guard 51.

However, in certain cases and notably for patients having a great corpulence, upon removing the perforator guide 10, the end of the tubular sleeve 50 may be located at the emergence of the incision for introducing the perforator guide, even within this incision, so that the surgeon cannot introduce the tape or its introducer into the sleeve.

In order to overcome this drawback, according to an alternative embodiment of the invention, the tubular sleeve 50 has a greater length than the length Lu of the useful portion of the perforator guide 10 as this results from FIG. 14. In this case, the tubular sleeve 50 then has a side aperture 52 which is provided at a distance from the free end of the tubular sleeve, less than or equal to and according to the illustrated example, substantially equal to the useful length Lu of the perforator guide. The side aperture 52 then allows the perforator guide 10 to be placed into the sleeve 50 by letting the tip 12 protrude at the end of said tubular sleeve.

Thus, after placing the tubular sleeve in the body of the patient, by means of the perforator guide, the free portion 53 of the tubular sleeve 50 generously juts out from the body of the patient so that once the perforator guide 10 is removed, the surgeon may easily introduce the introducer or an implant into the tubular sleeve 50.

1. A device for placing a reinforcing tape (4) in a tissue of the human body, comprising an introducer (1) which has:
an elongated flexible body (2),
at each of both ends of the body, pulling means (5),
between both ends, a cavity (3) for receiving the tape (4),
and, at the cavity, means for cutting (7) the body (2) into two portions separable by pulling exerted on the pulling means (5),
characterized in that the cutting means (7) comprise at least one aperture (8, 8₁) provided in that wall of the cavity (3) and intended for the passage of a cutting tool.

2. The device according to claim 1, characterized in that the aperture (8) constituent of the cutting means (7), extends transversally to the longitudinal axis (Δ) of the cavity (3) and affects more than half of the circumference of the wall of the cavity (3).

3. The device according to claim 1, characterized in that the aperture (8) constituent of the cutting means (7), is adapted for allowing the placement of the tape (4) in the cavity.

4. The device according to claim 1, characterized in that the cutting means (7) comprise at least two apertures (8₁) positioned facing each other.

5. The device according to claim 1, characterized in that the wall of the cavity has a series of perforations (P) for sterilization.

6. The device according to claim 1, characterized in that the pulling means (5) comprise semi-rigid needles integral with the ends of the elongated body (2).

7. The device according to claim 1, characterized in that it comprises a tape positioned inside the cavity and being free.

8. The device according to claim 1, characterized in that it further comprises an elongated perforator guide (10) or trocar, an end of which is intended to be introduced into the body of a patient and the other end of which (13) is provided with a handle (14).

9. The device according to claim 8, characterized in that the perforator guide (10) has an arcuate shape in a plane.

10. The device according to claim 9, characterized in that the arcuate portion (15) of the perforator extends over an angular sector larger than 140° and preferably less than 180° and more preferably between 150° and 170°.

11. The device according to claim 9, characterized in that the arcuate portion (15) of the perforator guide (10) then has a radius of curvature (R) between 30 mm and 60 mm, and preferably between 40 mm and 50 mm for the portion of the perforator guide extending between the handle and the end intended to be introduced into the body of the patient.

12. The device according to claim 8, characterized in that the perforator guide (10) has a helicoidal shape at its end (17) opposite to the handle (14) or distal end.

13. The device according to claim 12, characterized in that the perforator guide (10) has the shape of a portion of a helicoidal coil (17) extending over an angle between 180° and 360°, and preferably between 255° and 270°.

14. The device according to claim 13, characterized in that the coil (17) of the perforator guide has a radius of curvature between 20 mm and 40 mm with a pitch between 15 mm and 25 mm.

15. The device according to claim 8, characterized in that it further comprises a removable tubular sleeve (50) with a complementary shape to that of the perforator guide, intended to be engaged onto the perforator guide and to remain in the body of the patient after removing the perforator guide (10) so as to define a tunnel for the passage of pulling means (5) of the introducer (1).

16. The device according to claim 15, characterized in that the tubular sleeve (50) has a length larger than the useful length (Lu) of the perforator guide and comprises a side aperture (52) for placing the perforator guide, the side aperture (52) being located at a distance from a free end of the sleeve (50), less than or equal to the useful length (Lu) of the perforator guide.
